(19) United States
(12) Patent Application Publication
(10) Pub. No.: US 2004/0260288 A1
(43) Pub. Date: Dec. 23, 2004

(54) Slipped capital femoral epiphysis fixation screw (SCFEFS)

(76) Inventor: Robert Earl Means JR., Baltimore, MD (US)

Correspondence Address: Robert Earl Means, Jr., 4215 Belvieu Ave., Baltimore, MD, 21215, US

(21) Appl. No.: 10/601,206

(22) Filed: Jun. 20, 2003

(51) Int. Cl. A61B 17/56

(52) U.S. Cl. 606/65

(57) ABSTRACT

The Slipped Capital Femoral Epiphyseal Fixation Screw is an Orthopaedic fixation devise for treatment of pediatric and adolescent femoral neck fractures through the epiphyseal plate. The unique innovation involves constructing a contracted anchoring fixation head at the end of a cannulated screw barrel. This fixation screw is designed to eliminate epiphyseal plate bridging by a threaded segment of a fixation screw, which is currently unavoidable with current fixation screws due to the inherently elongated thread lengths.

### CROSS REFERENCE

My invention claims the benefit of the application described as the cannulated fixation screw, identified by the application number: 712414

With the filing date: Jun. 10, 1991

### BACKGROUND

This invention is classified as an Orthopaedic fixation devise used to maintain fractured bone positions frequently after trauma and/or manipulation.

The Slipped Capital Femoral Epiphysis Fixation Screw is a design that addresses the unique problem of fixating a pediatric hip fracture while allowing continued growth at the epiphyseal plate.

### SUMMARY OF INVENTION

The Slipped Capital Femoral Epiphysis Fixation Screw is an innovation addressing the problem of appropriate screw thread length necessary to both fixate a Salter classified epiphyseal plate fracture or subluxation, and eliminate growth obstruction at the epiphyseal plate site.

Previous lower extremity long bone fixation screws are designed with thread lengths greater than 4 threads. The standard doctrine in fixation of SCFE is to prevent screw thread bridging of the epiphyseal plate. Translation of thread length across the epiphyseal plate prevents continued growth, and subsequent elongation of the femoral neck in accordance with standard development of the hip.

The SCFEFS is a unique screw with a characteristically short screw length. The screw head is comprised of 2 to 3 anchoring threads at the end of a cannulated barrel, flanked on both ends of the anchoring threads by ½ cutting threads.

The short thread lengths enables placement of the screw head within the confines of the epiphysis thus allowing continued epiphyseal plate bone production and femoral neck growth.

Proposed screw lengths will generally range in the 60 mm to 100 mm range.

Current conventional screw barrel diameters are 4.5 mm and 6.0 mm, suitable for small and large frame pediatric patients respectively.

There are current screws manufactured with short thread lengths, but have short screw lengths and small diameters for small bone fixation applications unsuitable for treatment of femoral head and neck fractures.

No current fixation screw has the dimensions of length and diameter necessary in the treatment of hip epiphyseal plate fractures while enabling fixation without obstructing epiphyseal growth patterns.

This appliance ensures secure fixation and unimpeded growth while the epiphyseal plate fracture healing is completed.

The cutting threads allow expedient screw removal when extraction is indicated.

### DESCRIPTION OF DRAWINGS

The figures depicted are three drawings showing the screw in cross section, enlarged anchoring head view, and the fixation screw as it would appear positioned in an anatomically correct drawing of a pediatric proximal femur.

FIG. 1. show the cross sectional view presenting a barreled shaft describing the inside and outside diameters, screw attachment head for facilitating insertion and removal of the screw with a screw driver, and the anchoring screw head.

FIG. 2. show an enlarged view of the anchoring screw head. Two to three full threads are proposed. A one half thread on both ends of the full length threads aids in advancing and retracting the screw for insertion and extraction as needed.

FIG. 3. show the pediatric proximal femur in coronal view. The epiphyseal plate is the site of subluxation or dislocation that can occur in Slipped Capital Femoral Epiphyseal pathology. In treating this problem; the epiphysis is fixed in place with anchoring threads, ideally without crossing the epiphyseal plate with a threaded barrel. This FIG. 3. show the fixation screws with the epiphysis anchored in place by the anchoring threads. The epiphyseal plate is traversed by smooth non-threaded barrel.

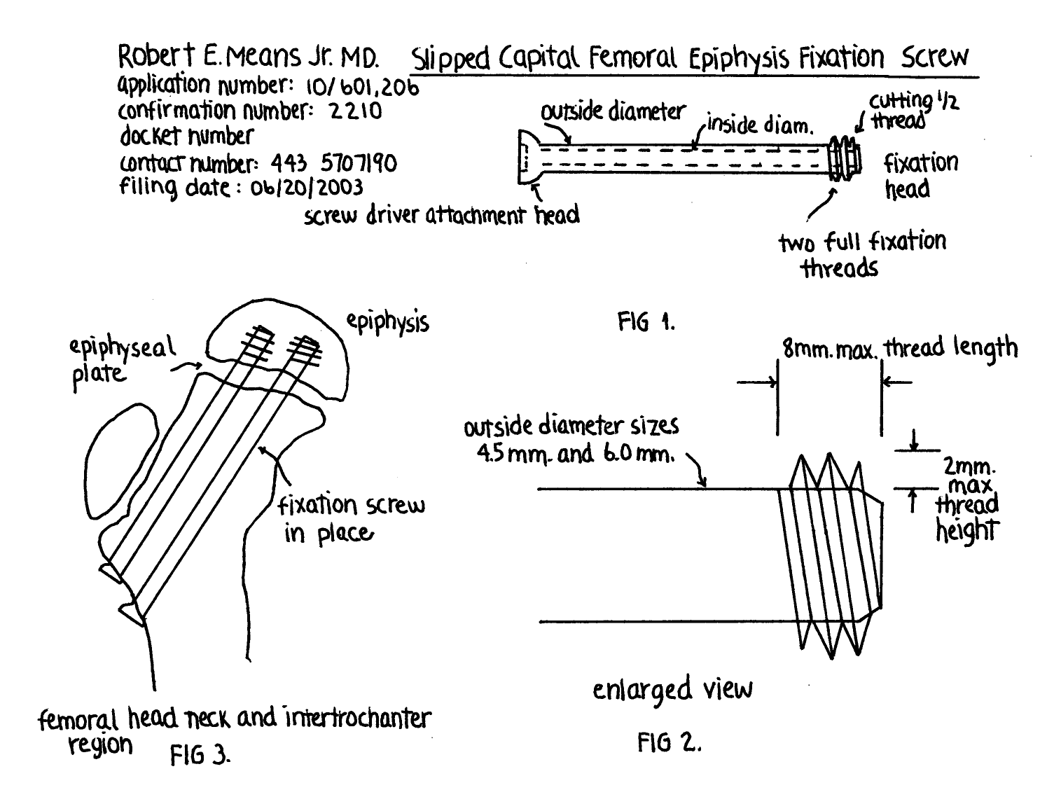

### DETAILED DESCRIPTION OF THE SCFEFS

This invention is an improvement of an established cannulated fixation screw.

The current long bone fixation screws do not address the unique situation and structure of the pediatric proximal femur fracture at the epiphyseal plate.

By combining a cannulated barrel with a contracted anchoring fixation head, fixation of a femoral neck fracture can be achieved without impeding femoral neck growth.

1. What I claim as my invention is the configuration of a contracted anchoring head on a cannulated fixation screw barrel that will allow fixation of a fractured neck of a pediatric proximal femur while eliminating the onset of growth arrest or fusion of the epiphyseal plate secondary to screw thread bridging of the plate.
